(19) United States
(12) Patent Application Publication
(10) Pub. No.: US 2018/0360695 A1
(43) Pub. Date: Dec. 20, 2018

(54) VALVE ARRANGEMENT FOR ENTERAL FEEDING SETS HAVING MULTIPLE FLUID SOURCES

(71) Applicant: AXIUM MTECH SA, Nyon (CH)

(72) Inventors: Michael Jedwab, Lausanne (CH); Nicolas Andrey, Echandens (CH)

(21) Appl. No.: 16/063,018

(22) PCT Filed: Dec. 12, 2016
(86) PCT No.: PCT/EP2016/080577

§ 371 Date: Jun. 15, 2018

Related U.S. Application Data
(60) Provisional application No. 62/268,193, filed on Dec. 16, 2015.

(51) Int. Cl. A61J 15/00; A61M 39/24; A61M 5/168

(52) U.S. Cl. CPC A61J 15/0092; A61M 5/16827; A61M 39/24

(57) ABSTRACT

A system can sequentially administer two fluids from a pair of containers (101, 102) to a patient. The system can have a delivery device (11) integrating two valve assemblies into a single device, and preferably each of the valve 102 assemblies has two one-way valves and a pump communication port (124, 125). For example, the valve assemblies may share one or more walls and/or one or more chambers with each other. Preferably the only exits from the housing are attached to input tubing, output tubing, and connections to the pump. Preferably the system uses a single pump (12). By reversing the pumping direction of the pump, liquid may be sequentially drawn from one container or the other. The valve assemblies can use slit valves (13d) to prevent free-flow of the fluids.

### BACKGROUND

The present disclosure relates generally to devices and methods for administering multiple fluids such as enteral feeding solutions. More specifically, the present disclosure is directed to a valve arrangement configured to sequentially administer multiple fluids.

When a patient is unable to eat normally, an infusion set can provide an enteral solution containing nutrition and optional medication to the patient. The infusion set can be used with a pump (e.g., a peristaltic pump) to regulate the amount and the rate at which the enteral solution is delivered from a reservoir to the patient.

Nutritional needs and hydration needs of patients fed enterally are often greater than can be supplied by commercially available enteral solution containers. To save time, caregivers such as nurses utilize administration sets that allow simultaneous hanging of two separate containers. Individually controlling the flow from each of these containers either adds complexity to the feeding pump or requires additional tubing length that can be tangled or kinked when used by mobile patients in a backpack.

Typically the amount of enteral solution administered to the patient must be precisely controlled, especially if the enteral solution contains potent compounds. In many enteral feeding systems, the engagement of the tube to a peristaltic pump controls the flow of fluid to the patient according to the speed of the peristaltic pump. Nevertheless, excess fluid can reach the patient due to gravity, which is known as free-flow and is not only undesirable but can be dangerous.

In infusion set configurations that include multiple separate containers, clamps on each of the lines between the individual fluid containers and the pump can allow manual selection of the fluid source. However, a drawback of this configuration is that the user must manually switch from one fluid source to the other fluid source. In most arrangements, this manual switching does not protect against free-flow of the enteral solution. Additionally, when using manual clamps, the pump is unable to identify which fluid source is being used. Furthermore, the clamps can lead to use errors, such as inadvertently clamping the wrong source or clamping both sources.

Two separated pump mechanisms can be used. For example, a delivery set can include a pump interface on both lines between the fluid container and a point of junction into one single line. A drawback of this system is that two independent pumping mechanisms on the pump make the system too heavy and bulky for mobile use.

A valve arrangement may be positioned external from the pump interface, but the tubing arrangement is complex (e.g., two pieces of tube above the pump, two pieces of tube below the pump, and one piece of tube after the tube junction). This complexity is an issue in mobile use, especially in backpack use, where tubes run the risk of being kinked and thereby preventing fluid from reaching the patient.

A fluid container actively selected by the pump can be used. For example, a delivery set arrangement can have a valve actuated by the pump that can selectively open a fluid path for each of the fluid container lines. However, this system requires an additional actuator inside the pump.

### SUMMARY

The present disclosure provides a valve arrangement for an enteral administration set having multiple fluid containers. A delivery device can integrate two or more valve assemblies into a single device, and preferably each of the valve assemblies has two one-way valves and a pump communication port. The one-way valves can be grouped such that the only exits from the delivery device are the input tubing, the output tubing, and the pump connection. The one-way valves can be configured so that reversing the motor rotation allows selective feeding from the first fluid bag or the second fluid bag.

Accordingly, in a general embodiment, the present disclosure provides a delivery device comprising a housing comprising a first inlet port, a second inlet port, an outlet port, a first pump communication port, and a second pump communication port. The delivery device further comprises a first one-way valve, a second one-way valve, a third one-way valve, and a fourth one-way valve. A first flow path extends from the first inlet port through the first one-way valve and the third one-way valve to the outlet port. A second flow path extends from the second inlet port through the second one-way valve and the fourth one-way valve to the outlet port.

In an embodiment of this delivery device, a flexible membrane is at least partially positioned within the housing, and the flexible membrane provides the first, second, third and fourth one-way valves. The flexible membrane can be one single membrane or can be formed by multiple individual membranes, for example two membranes or four membranes.

In an embodiment, the housing comprises a first chamber, a second chamber, a third chamber, a fourth chamber, and an administration chamber that define at least part of the first flow path. The first chamber can be accessible from the exterior through the first inlet port, the second chamber can be accessible from the exterior through the first pump connection port, the third chamber can be accessible from the exterior through the second inlet port, the fourth chamber can be accessible from the exterior through the second pump connection port, and the administration chamber can be accessible from the exterior through the outlet port.

In another embodiment, the present disclosure provides a system comprising a pump comprising a first passage that leads to the first pump communication port of the delivery device and a second passage that leads to the second pump communication port of the delivery device. The pump can be a single pump that is the only pump connected to the delivery device. The pump is configured to pump in a first direction to direct the first fluid through the first flow path while at least one of the one-way valves is closed to prevent the second fluid from moving through the second flow path. The pump is configured to pump in a second direction opposite to the first direction to direct the second fluid through the second flow path while at least one of the one-way valves is closed to prevent the first fluid from moving through the first flow path.

In another embodiment, the present disclosure provides a method of sequentially administering a first fluid and a second fluid to a patient using a single pump connected to a housing of a delivery device. The delivery device comprises a first one-way valve, a second one-way valve, a third one-way valve, and a fourth one-way valve within the housing. The method comprises operating the pump in one pumping direction to direct the first fluid from a first container in a first flow path through the delivery device and out of an administration port of the housing of the delivery device. The first flow path comprises the first one-way valve and the third one-way valve. Simultaneously, the second and fourth one-way valves are closed to prevent the second fluid from moving through the second flow path, and the first and third one-way valves are open.

The method further comprises reversing the pumping direction of the pump. The reversing of the pumping direction prevents movement of the first fluid through the delivery device. The reversing of the pumping direction also directs the second fluid in a second flow path through the delivery device and out of the same administration port of the delivery device that the first fluid used to exit the housing of the delivery device. The second flow path comprises the second one-way valve and the fourth one-way valve. Simultaneously, the first and third one-way valves are closed to prevent the first fluid from moving through the first flow path, and the second and fourth one-way valves are open.

In another embodiment, the present disclosure provides a method of making a delivery device configured to connect to a single pump and sequentially administer a first fluid and a second fluid to a patient. The method comprises forming a housing that contains a first flow path extending from a first inlet port on the housing to an outlet port on the housing. The housing contains a second flow path extending from a second inlet port on the housing to the outlet port on the housing. The method comprises positioning a first one-way valve, a second one-way valve, a third one-way valve, and a fourth one-way valve within the housing. The first one-way valve and the third one-way valve are positioned in the first flow path; and the second one-way valve and the fourth one-way valve are positioned in the second flow path. Any number of outlet ports can be used, and the present disclosure is not limited to a specific number of outlet ports.

In an embodiment, the positioning of the first, second, third and fourth one-way valves within the housing comprises positioning one or more flexible membranes comprising the first, second, third and fourth one-way valves at least partially within the housing.

In another embodiment, the present disclosure provides a method of making a system configured to sequentially administer a first fluid and a second fluid to a patient. The method comprises connecting a pump to the housing of the delivery device. The pump is preferably a single pump that is the only pump connected to the delivery device.

In an embodiment of this method, the method comprises connecting to the first inlet port a first tube that leads to (i) a first container that holds the first fluid and/or (ii) a first connector configured to connect to a first container that holds a first fluid. The method can further comprise connecting to the second inlet port a second tube that leads to (i) a second container that holds the second fluid and/or (ii) a second connector configured to connect to a second container that holds a second fluid. Nevertheless, any number of connectors and any number of containers can be used, and the present disclosure is not limited to a specific number of connectors or a specific number of containers. The method preferably comprises connecting an administration tube to the outlet port, and the administration tube leads to an enteral feeding tube.

An advantage of one or more embodiments provided by the present disclosure is to automatically select a fluid container of an enteral administration set having a plurality of fluid containers.

Another advantage of one or more embodiments provided by the present disclosure is an enteral administration set having a plurality of fluid containers that uses one single pumping mechanism.

A further advantage of one or more embodiments provided by the present disclosure is to reduce pump complexity by eliminating the requirement for an additional actuator to select the fluid container.

Yet another advantage of one or more embodiments provided by the present disclosure is to increase safety by reducing the risk of tubing being tangled or kinked.

Another advantage of one or more embodiments provided by the present disclosure is to increase safety by preventing free-flow situations.

Additional features and advantages are described herein and will be apparent from the following Detailed Description and the Figures.

### BRIEF DESCRIPTION OF THE FIGURES

FIG. 1 shows a schematic diagram of a sequential administration system provided by the present disclosure.

FIG. 2 shows a schematic diagram of a sequential administration system provided by the present disclosure, illustrating mechanical connections (left side) and fluid connections (right side).

FIG. 3 shows a side cross-section view of an embodiment of a delivery device provided by the present disclosure.

FIG. 4 shows an exploded view of the embodiment of the delivery device depicted in FIG. 3.

### DETAILED DESCRIPTION

As used in this disclosure and the appended claims, the singular forms “a,” “an” and “the” include plural referents unless the context clearly dictates otherwise. Thus, for example, reference to “a fluid” or “the fluid” includes two or more fluids.

The words “comprise,” “comprises” and “comprising” are to be interpreted inclusively rather than exclusively. Likewise, the terms “include,” “including” and “or” should all be construed to be inclusive, unless such a construction is clearly prohibited from the context.

Nevertheless, the devices and apparatuses disclosed herein may lack any element that is not specifically disclosed. Thus, a disclosure of an embodiment using the term “comprising” includes a disclosure of embodiments “consisting essentially of” and “consisting of” the components identified. Similarly, the methods disclosed herein may lack any step that is not specifically disclosed herein. Thus, a disclosure of an embodiment using the term “comprising” includes a disclosure of embodiments “consisting essentially of” and “consisting of” the steps identified.

The term “and/or” used in the context of “X and/or Y” should be interpreted as “X,” or “Y,” or “X and Y.” Where used herein, the terms “example” and “such as,” particularly when followed by a listing of terms, are merely exemplary and illustrative and should not be deemed to be exclusive or comprehensive. Any embodiment disclosed herein can be combined with any other embodiment disclosed herein unless explicitly stated otherwise.

As used herein, “about” and “approximately” are understood to refer to numbers in a range of numerals, for example the range of −10% to +10% of the referenced number, preferably within −5% to +5% of the referenced number, more preferably within −1% to +1% of the referenced number, most preferably within −0.1% to +0.1% of the referenced number.

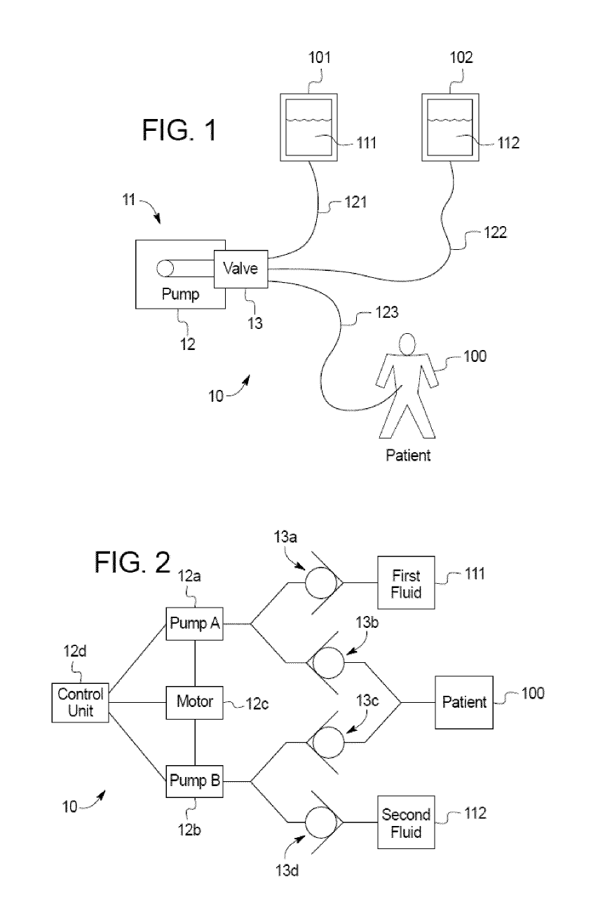

As shown in FIG. 1, an aspect of the present disclosure is a system 10 which may be used to sequentially administer a first fluid 111 and a second fluid 112. The system 10 can comprise a first container 101 configured to hold the first fluid 111 and a second container 102 configured to hold the second fluid 112.

The system 10 can comprise a pump 12 and can comprise a delivery device 11 comprising a valve arrangement 13. In an embodiment, the pump 12 and the delivery device 11 comprising the valve arrangement 13 are provided as a single unitary device, although in other embodiments the pump 12 and the delivery device 11 comprising the valve arrangement 13 are separate devices not integral with each other.

In a preferred embodiment, the system 10 comprises a first tube 121 in fluid communication with the first container 101 and the delivery device 11, a second tube 122 in fluid communication with the second container 102 and the delivery device 11, and an administration tube 123 in fluid communication with the delivery device 11. The first tube 121 can convey the first fluid 111 from the first container 101 to the delivery device 11, the second tube 122 can convey the second fluid 112 from the second container 102 to the delivery device 11, and the administration tube 123 can sequentially convey the first and second fluids 111,112 from the delivery device 11 to a free end of the administration tube 123 (e.g., the opposite end from the end connected to delivery device 11). In some embodiments, the first tube 121 and/or the second tube 122 are part of the delivery device 11. Each of the first, second and administration tubes 121-123 can be made of a flexible material such as polyvinyl chloride or silicone rubber.

The free end of the administration tube 123 can connect to a catheter, an enteral feeding tube, or another device configured to administer at least one of the first fluid 111 or the second fluid 112 to a patient 100. The third tube 123 can connect directly to such an administration device or may use an intermediate connector component.

The right side of the schematic diagram in FIG. 2 represents the flow of the first and second fluids 111,112. The valve arrangement 13 can comprise a first one-way valve 13a, second one-way valve 13b, a third one-way valve 13c, and a fourth one-way valve 13d. The first one-way valve 13a receives the first fluid 111 from the first tube 121, the pump 12 conveys the first fluid 111 from the first one-way valve 13a to the third one-way valve 13c, and the third one-way valve 13c conveys the first fluid 111 to the administration tube 123. As discussed in more detail later herein, while the first fluid 111 moves through this first flow path, the fourth one-way valve 13d simultaneously is closed to prevent flow of the second fluid 112 through the delivery device 11.

The fourth one-way valve 13d receives the second fluid 112 from the second tube 122, the pump 12 conveys the second fluid 112 from the fourth one-way valve 13d to the second one-way valve 13b, and the second one-way valve 13b conveys the second fluid 112 to the administration tube 123. As discussed in more detail later herein, while the second fluid 112 moves through this second flow path, the first one-way valve 13a simultaneously is closed to prevent flow of the first fluid 111 through the delivery device 11.

Each of the first, second, third and fourth one-way valves 13a-13d can be any one-way valve known to one skilled in this art. Specifically, a “one-way valve” is any valve that allows fluid transfer in only one direction. Each of the first, second, third and fourth one-way valves 13a-13d can have a zero cracking (threshold) pressure or a non-zero cracking (threshold) pressure; the cracking (threshold) pressure is the inlet pressure at which the first indication of flow occurs. In this regard, a one-way valve having a zero cracking pressure allows fluid to flow freely in the desired direction, and a one-way valve having a non-zero cracking pressure (positive cracking pressure) provides resistance against the fluid flowing therethrough in the desired direction. In the other direction, the cracking pressure may be thought of as infinite because no fluid can flow in this direction regardless of the pressure difference. As discussed in more detail later herein, the cracking pressures of the first, second, third and fourth one-way valves 13a-13d can be specifically established to prevent free-flow through the valve arrangement 13.

The system 10 can comprise an anti-free flow mechanism separate from the valve arrangement 13. For example, the system 10 can comprise a fifth one-way valve 13e and a sixth one-way valve 13g upstream from the valve arrangement 13. The fifth one-way valve 13f can be associated with the first container 101 and/or the first tube 121, and the sixth one-way valve 13g can be associated with the second container 102 and/or the second tube 122. In some embodiments, the fifth and sixth one-way valves 13f,13g are part of the delivery device 11; in other embodiments, the fifth and sixth one-way valves 13f,13g are external to the delivery device 11.

Additionally or alternatively, the system 10 can comprise a seventh one-way valve 13h downstream from the valve arrangement 13. The seventh one-way valve 13h can be associated with the administration tube 123. In some embodiments, the seventh one-way valve 13h is part of the delivery device 11; in other embodiments, the seventh one-way valve 13h is external to the delivery device 11.

The separate anti-free flow mechanism can be used when one or more of the first, second, third and fourth one-way valves 13a-13d (e.g., one, two, three or all four of these one-way valves) has a zero cracking pressure or a very low cracking pressure (e.g., less than about 0.1 bars). Preferably the separate anti-free flow mechanism, if present, only comprises one of (i) the fifth and sixth one-way valves 13f,13g (both of these one-way valves simultaneously present) or (ii) the seventh one-way valve 13h; but in some embodiments all three of the fifth, sixth and seventh one-way valves 13f-13h are present.

The left side of the schematic diagram in FIG. 2 represents the mechanical connections of the delivery device 11. The pump 12 can comprise a first pump passage 12a and a second pump passage 12b. The delivery device 11 can comprise a first pump communication port 124 and a second pump communication port 125 in fluid communication with the first pump passage 12a and the second pump passage 12b, respectively.

In embodiments where the pump 12 is a peristaltic pump, the first pump passage 12a is preferably provided by a first pump pipe, and the second pump passage 12b is preferably provided by a second pump pipe, although in some embodiments a single pump pipe is connected to both of the first and second pump communication ports 124,125. The present disclosure is not limited to a specific embodiment of the first and second pump passages 12a,12b. For example, if the pump 12 is a volumetric pump, the first and second pump passages 12a,12b can merely be openings that respectively connect the first and second pump communication ports 124,125 of the valve arrangement 13 directly to a chamber of the pump 12.

The pump 12 is configured such that the first pump passage 12a provides discharge while the second pump passage 12b simultaneously provides suction, and the first pump passage 12a provides suction while the second pump passage 12b simultaneously provides discharge. Thus the first and second pump passages 12a,12b do not ever provide the same direction of displacement. The pump 12 can be a positive displacement pump, non-limiting examples of which include a peristaltic pump, a gear pump, a lube pump, an impeller pump, and a piston pump. The pump 12 can be any type of pump capable of pumping gas or liquid at controlled flow rates in both directions in alternating fashion and which is suitable for clinical applications.

In an embodiment, the system 10 can include a control unit 12d for one or more of data entry, information display, alarm signaling, and/or manual control of the pump 12. The control unit 12d may include a microprocessor for controlling and activating the pump 12. The pump 12 can comprise a motor 12c that can rotate a shaft and/or operate another mechanism to initiate pumping.

In embodiments where the pump 12 comprises a shaft, the direction of rotation of the shaft can establish the direction of pumping. For example, the shaft of the motor 12c can rotate one direction such that the first pump passage 12a provides suction while the second pump passage 12b simultaneously provides discharge. The shaft of the motor 12c can rotate the opposite direction such that the first pump passage 12a provides discharge while the second pump passage 12b simultaneously provides suction. The motor 12c can be controlled by the control unit 12d; for example, the control unit 12d can control and coordinate the direction of rotation of the motor 12c. Nevertheless, the present disclosure is not limited to a specific embodiment of the pump 12, and the pump 12 can operate using a mechanism alternative or additional to rotation.

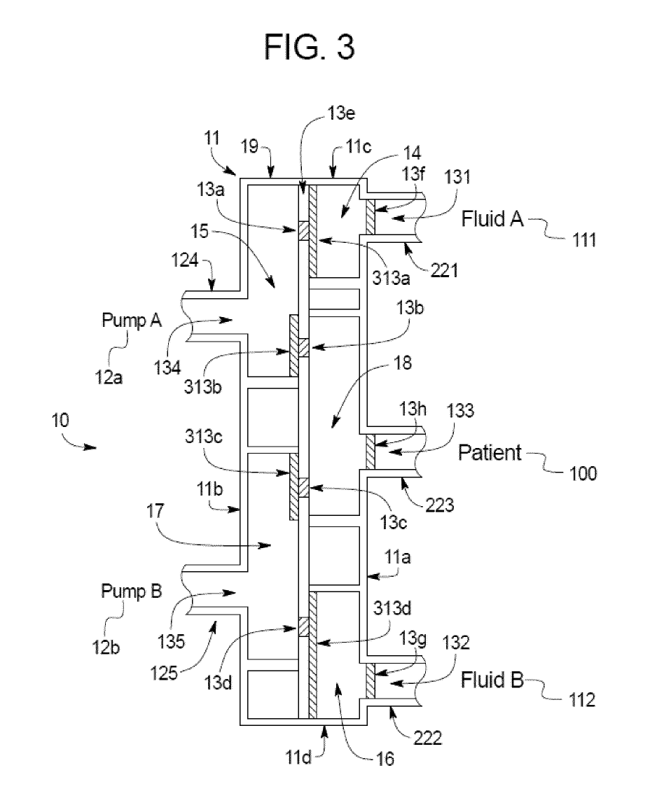

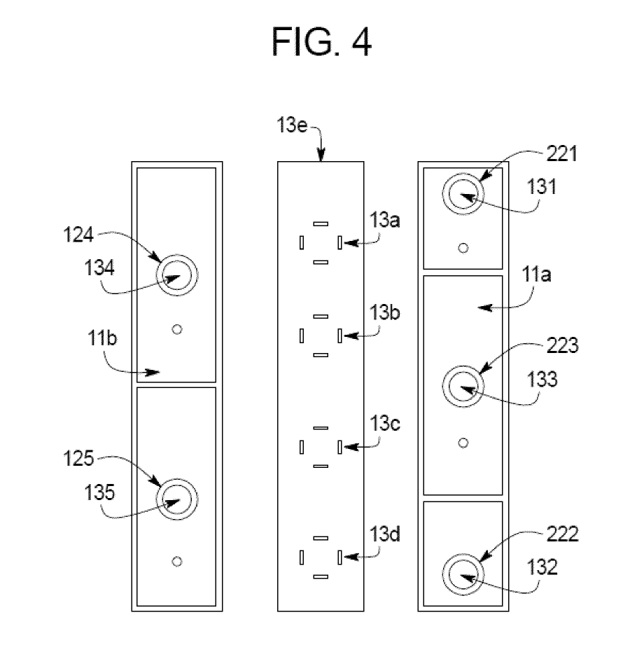

A particularly preferred embodiment of the delivery device 11 is shown in FIGS. 3 and 4. The delivery device 11 can comprise a housing 19 that contains the valve arrangement 13. The housing 19 can be made of a metal and/or a plastic, such as acrylonitrile butadiene styrene (ABS), polycarbonate, polyvinyl chloride (PVC), an acrylic material, or methyl methacrylate-acrylonitrile-butadiene-styrene (MABS).

The valve arrangement 13 can comprise a flexible membrane 13e positioned in the housing 19. The first, second, third and fourth one-way valves 13a-13d can each be slit valves. For example, each of the first, second, third and fourth one-way valves 13a-13d can be a plurality of slits (FIG. 4) which are completely closed in a resting state and thus do not permit flow of liquid through the flexible membrane 13e. Nevertheless, each of the first, second, third and fourth one-way valves 13a-13d is not limited to a specific type of valve and may be any suitable valve known to one skilled in this art.

The flexible membrane 13e can be one single membrane or can be formed by multiple individual membranes. A non-limiting example of a multi-membrane embodiment includes a first flexible membrane that provides one or more of the first, second, third and fourth one-way valves 13a-13d (e.g., the first and second one-way valves 13a,13b) and includes a second flexible membrane that provides the remainder of the first, second, third and fourth one-way valves 13a-13d (e.g., the third and fourth one-way valves 13c,13d). Another non-limiting example of a multi-membrane embodiment includes four flexible membranes, each of which provides a corresponding one of the first, second, third and fourth one-way valves 13a-13d. Similarly, the housing 19 can be formed of one single piece of material or multiple pieces of material connected together.

The flexible membrane 13e can be made of a resilient flexible material, for example a sterilizable material such as silicon, rubber, polyurethane, or any other suitable material. Preferably the flexible membrane 13e is one single piece, for example a single piece of material or a plurality of pieces of material connected together.

In embodiments using slit valves, the flexible membrane 13e can be designed so that the slits will open only when the pressure differential over the flexible membrane 13e exceeds a threshold. This design can prevent undesired free-flow of the first and second fluids 111,112 from the first and second containers 101,102 which in a clinical setting are typically placed on a stand of a height of about 2 to about 3 meters.

The first pump passage 12a can be connected to a first pump communication port 124 provided by the housing 19 of the delivery device 11, and the second pump passage 12b can be connected to a second pump communication port 125 provided by the housing 19 of the delivery device 11. The first tube 121 can be connected to a first inlet port 221 provided by the housing 19 of the delivery device 11, the second tube 122 can be connected to a second inlet port 222 provided by the housing 19 of the delivery device 11, and the administration tube 123 can be connected to one or more outlet ports 223 (“the outlet port 223”) provided by the housing 19 of the delivery device 11. The present disclosure is not limited to a specific number of the one or more outlet ports 223.

In an embodiment, one or more of the first and second inlet ports 221,222 and the outlet port 223 are provided by a first wall 11a of the delivery device 11. One or more of the first and second pump communication ports 124,125 can be provided by a second wall 11b of the delivery device 11. Alternatively or additionally, one or more of the first and second pump communication ports 124,125 can be provided by the first wall 11a of the delivery device 11.

The first wall 11a can comprise a first inlet 131 circumscribed by the first inlet port 221 and extending through the first wall 11a such that the first fluid 111 can travel into the interior of the delivery device 11. The first wall 11a can comprise a second inlet 132 circumscribed by the second inlet port 222 and extending through the first wall 11a such that the second fluid 112 can travel into the interior of the delivery device 11. The first wall 11a can comprise an outlet 133 circumscribed by the outlet port 223 and extending through the first wall 11a such that one of the first and second fluids 111,112 can emerge from the interior of the delivery device 11 into the exterior. The second wall 11b can comprise a first opening 134 circumscribed by the first pump communication port 124 and extending through the second wall 11b and can comprise a second opening 135 circumscribed by the second pump communication port 125 and extending through the second wall 11b.

In an embodiment where the fifth and sixth one-way valves 13e,13g are present, the fifth one-way valve 13f can be associated with the first inlet 131, and the sixth one-way valve 13g can be associated with the second inlet 132. In an embodiment where the seventh one-way valve 13h is present, the seventh one-way valve 13h can be associated with the outlet 133.

The first wall 11a is preferably on an opposite side of the delivery device 11 from the second wall 11b. For example, the housing 19 of the delivery device 11 can comprise a third wall 11c and/or a fourth wall 11d that connect the first wall 11a to the second wall 11b. The flexible membrane 13e can extend from the third wall 11c to the fourth wall 11d. In an embodiment, the ends of the flexible membrane 13e are fixedly attached to the third wall 11c and/or the fourth wall 11d.

In a preferred embodiment, the first wall 11a, the third wall 11c and the flexible membrane 13e define at least part of a first chamber 14 positioned between the first inlet 131 and the first one-way valve 13a. The second wall 11b, the third wall 11c and the flexible membrane 13e can define at least part of a second chamber 15 positioned between the first one-way valve 13a and the first opening 134. The first wall 11a, the fourth wall 11d and the flexible membrane 13e can define at least part of a third chamber 16 positioned between the second inlet 132 and the fourth one-way valve 13d. The second wall 11b, the fourth wall 11d and the flexible membrane 13e can define at least part of a fourth chamber 17 positioned between the fourth one-way valve 13d and the second opening 135. The first wall 11a and the flexible membrane 13e can define at least part of an administration chamber 18 positioned between the first wall 11a and the second and third one-way valves 13b,13c. The administration chamber 18 is preferably positioned between the first chamber 14 and the third chamber 16.

The delivery device 11 can comprise at least two valve assemblies. For example, the first chamber 14, the second chamber 15 and the administration chamber 18 can form at least part of a first valve assembly; and/or the third chamber 16, the fourth chamber 17 and the administration chamber 18 can form at least part of a second valve assembly. The first valve assembly and the second valve assembly can share at least one chamber (e.g., the administration chamber 18) and can share at least one wall (e.g., the first wall 11a and/or the second wall 11b).

In an embodiment, the housing 19 contains one or more supports associated with the flexible membrane 13e and configured to prevent the flexible membrane 13e from deforming in an opposite flow direction. The one or more supports can thereby prevent back flow. For example, a section of the flexible membrane 13e in which one of the first, second, third and fourth one-way valves 13a-13d is positioned can seal against a corresponding support when the corresponding pressure differential is below the threshold of the valve. The valve sealing against the corresponding support can close the valve and prevent fluid flow in the direction opposite to the desired direction. In this regard, the supports ensure that each of the first, second, third and fourth one-way valves 13a-13d can only deform in one direction (the direction of desired direction of fluid flow) and cannot deform both directions.

Each of the supports can be any structure that prevents the corresponding valve from deforming in a direction opposite to the desired direction of fluid flow, for example a plate, a tube or other structure pressed against the valve, and the support is not limited to a specific structure. Preferably the support has one or more openings that allow the fluid to flow therethrough to the corresponding valve.

However, in some embodiments, one or more of the first, second, third and fourth one-way valves 13a-13d sufficiently close by collapsing inward when the corresponding pressure differential is below the threshold of the valve, and these one or more collapsing one-way valves do not use a support.

FIG. 3 generally illustrates a non-limiting embodiment which uses supports to prevent the corresponding valve from deforming in a direction opposite to the desired direction of fluid flow. For example, the delivery device 11 can comprise a first support 313a that prevents the first one-way valve 13a from deforming toward the first chamber 14 and thus prevents fluid flow from the second chamber 15 into the first chamber 14. The delivery device 11 can comprise a second support 313b that prevents the second one-way valve 13b from deforming toward the second chamber 15 and thus prevents fluid flow from the administration chamber 18 into the second chamber 15. The delivery device 11 can comprise a third support 313c that prevents the third one-way valve 13c from deforming toward the fourth chamber 17 and thus prevents fluid flow from the administration chamber 18 into the fourth chamber 17. The delivery device 11 can comprise a fourth support 313d that prevents the fourth one-way valve 13d from deforming toward the third chamber 16 and thus prevents fluid flow from the fourth chamber 17 into the third chamber 16.

Operation of the system 10 can be performed as follows hereafter. During operation of the system 10, only one of the first fluid 111 or the second fluid 112 is flowing at a given time, and the other fluid is prevented from flowing by the one-way valves 13a-13h.

The first fluid 111 can flow into the first tube 121 and the first chamber 14, for example by gravity and/or pumping. The first pump passage 12a can exert suction at the first opening 134 to reduce the pressure in the second chamber 15 to thereby increase the ratio of the pressure in the first chamber 14 to the pressure in the second chamber 15. When this pressure differential between the first chamber 14 and the second chamber 15 exceeds a threshold of the first one-way valve 13a, e.g. the pressure in the first chamber 14 is greater than the pressure in the second chamber 15 by at least the threshold of the first one-way valve 13a, the first fluid 14 can travel from the first chamber 14 through the first one-way valve 13a into the second chamber 15.

Specifically, the section of the flexible membrane 13e comprising the first one-way valve 13a (which can be a slit valve) can be deformed, stretched and/or deflected by the pressure differential such that the slits of the first one-way valve 13a widen and open to allow flow of the first fluid 111 from the first chamber 14 through the first one-way valve 13a into the second chamber 15. Moreover, the suction at the first opening 134 keeps the second one-way valve 13b closed (e.g., sealed against the second support 313b), and thus the second one-way valve 13b prevents the first fluid 111 from travelling from the second chamber 15 to the administration chamber 18.

As noted above, the pump 12 is configured such that the first pump passage 12a provides suction while the second pump passage 12b simultaneously provides discharge. As a result, when the first pump passage 12a exerts suction at the first opening 134 as discussed in the preceding paragraph, the second pump passage 12b simultaneously provides discharge at the second opening 135. This discharge into the fourth chamber 17 increases the pressure in the fourth chamber 17 and thus ensures that the second fluid 112 cannot flow through the fourth one-way valve 13d.

In this regard, the fourth one-way valve 13d prevents flow of the second fluid 112 therethrough when the pressure differential between the third chamber 16 and the fourth chamber 17 is less than the threshold of the fourth one-way valve 13d, e.g. the pressure in the third chamber 16 is not greater than the pressure in the fourth chamber 17 by at least the threshold of the fourth one-way valve 13d. For example, in embodiments where the fourth support 313d is used, the discharge into the fourth chamber 17 by the pump 12 presses the fourth one-way valve 13d against the fourth support 313d to hold the fourth one-way valve 13d closed. In an embodiment where the fourth one-way valve 13d is a slit valve, the slits are held closed and cannot be deformed into the open position by the second fluid 112.

The pump 12 can then pull the first fluid 111 into the first pump passage 12a from the second chamber 15 and then push the first fluid 111 through the second pump passage 12b into the fourth chamber 17. The first fluid 111 entering the fourth chamber 17 and continuing to be pumped into the fourth chamber 17 can increase the pressure in the fourth chamber 17 and thus open the third one-way valve 13c. Furthermore, this pressure in the fourth chamber 17 can continue to maintain the fourth one-way valve 13d in the closed position, and thus the fourth one-way valve 13d can continue to prevent the second fluid 112 from flowing through the delivery device 11.

For example, the first fluid 111 entering the fourth chamber 17 can increase the ratio of the pressure in the fourth chamber 17 to the pressure in the administration chamber 18. When this pressure differential between the fourth chamber 17 and the administration chamber 18 exceeds the threshold of the third one-way valve 13c, e.g. the pressure in the fourth chamber 17 is greater than the pressure in the administration chamber 18 by at least the threshold of the third one-way valve 13c, the first fluid 111 can travel from the fourth chamber 17 through the third one-way valve 13c into the administration chamber 18. Specifically, the section of the flexible membrane 13e comprising the third one-way valve 13c (which can be a slit valve) can be deformed, stretched and/or deflected by the pressure differential such that the slits of the third one-way valve 13c widen and open to allow flow of the first fluid 111 from the fourth chamber 17 through the third one-way valve 13c into the administration chamber 18.

Continued pumping by the pump 12 can force the first fluid 111 from the administration chamber 18 into the administration tube 123 and then onward to the patient 100. The continued pumping keeps the pressure differential between the third chamber 16 and the fourth chamber 17 at less than the threshold of the fourth one-way valve 13d, e.g. the pressure in the third chamber 16 is not greater than the pressure in the fourth chamber 17 by at least the threshold of the fourth one-way valve 13d. Therefore, the delivery device 11 can form a first flow path that goes through the first one-way valve 13a, then through the pump 12, and then through the third one-way valve 13c, while simultaneously the second one-way valve 13b and the fourth one-way valve 13d are closed to prevent flow of the second fluid 112 through the delivery device 11.

Then the pumping direction of the pump 12 can be reversed. The pumping direction of the pump 12 may be reversed manually. Additionally or alternatively, the pumping direction of the pump 12 may be reversed automatically at selected times by the control unit 12d (e.g., a first pumping direction is employed for a first predetermined time period, then a second pumping direction opposite to the first direction is employed for a second predetermined time period, then the first pumping direction is employed for a third predetermined time period, etc.). Furthermore, the pumping direction of the pump 12 may be reversed a number of times by the control unit 12d (e.g., the pumping direction is reversed a predetermined number of times before stopping the pump 12).

The reversed pumping direction of the first pump 12 can exert suction at the second opening 135 to reduce the pressure in the fourth chamber 17 to thereby increase the ratio of the pressure in the third chamber 16 to the pressure in the fourth chamber 17. When this pressure differential between the third chamber 16 and the fourth chamber 17 exceeds the threshold of the fourth one-way valve 13d, e.g. the pressure in the third chamber 16 is greater than the pressure in the fourth chamber 17 by at least the threshold of the fourth one-way valve 13d, the second fluid 112 can travel from the third chamber 16 through the fourth one-way valve 13d into the fourth chamber 17.

Specifically, the section of the flexible membrane 13e comprising the fourth one-way valve 13d (which can be a slit valve) can be deformed, stretched and/or deflected by the pressure differential such that the slits of the fourth one-way valve 13d widen and open to allow flow of the second fluid 112 from the third chamber 16 through the fourth one-way valve 13d into the fourth chamber 17. Moreover, the suction at the second opening 135 keeps the third one-way valve 13c closed (e.g., sealed against the third support 313c), and thus the third one-way valve 13c prevents the second fluid 112 from travelling from the fourth chamber 17 to the administration chamber 18.

As noted above, the pump 12 is configured such that the first pump passage 12a provides discharge while the second pump passage 12b simultaneously provides suction. As a result, when the second pump passage 12b exerts suction at the second opening 135 as discussed in the preceding paragraph, the first pump passage 12a simultaneously provides discharge at the first opening 134. This discharge into the second chamber 15 increases the pressure in the second chamber 15 and thus ensures that the first fluid 111 cannot flow through the first one-way valve 13a.

The pump 12 can then pull the second fluid 112 into the second pump passage 12b from the fourth chamber 17 and then push the second fluid 112 through the first pump passage 12a into the second chamber 15. The second fluid 112 entering the second chamber 15 can increase the pressure in the second chamber 15 and thus open the second one-way valve 13b. Furthermore, this pressure in the second chamber 15 can continue to maintain the first one-way valve 13a in the closed position, and thus the first one-way valve 13a can continue to prevent the first fluid 111 from flowing through the delivery device 11.

For example, the second fluid 112 entering the second chamber 15 can increase the ratio of the pressure in the second chamber 15 to the pressure in the administration chamber 18. When this pressure differential between the second chamber 15 and the administration chamber 18 exceeds the threshold of the second one-way valve 13b, e.g. the pressure in the second chamber 15 is greater than the pressure in the administration chamber 18 by at least the threshold of the second one-way valve 13b, the second fluid 112 can travel from the second chamber 15 through the second one-way valve 13b into the administration chamber 18. Specifically, the section of the flexible membrane 13e comprising the second one-way valve 13b (which can be a slit valve) can be deformed, stretched and/or deflected by the pressure differential such that the slits of the second one-way valve 13b widen and open to allow flow of the second fluid 112 from the second chamber 15 through the second one-way valve 13b into the administration chamber 18.

Continued pumping by the pump 12 can force the second fluid 112 from the administration chamber 18 into the administration tube 123 and then onward to the patient 100. The continued pumping keeps the pressure differential between the first chamber 14 and the second chamber 15 at less than the threshold of the first one-way valve 13a, e.g. the pressure in the first chamber 14 is not greater than the pressure in the second chamber 15 by at least the threshold of the first one-way valve 13a. Therefore, the delivery device 11 can form a second flow path that goes through the fourth one-way valve 13d, then through the pump 12, and then through the second one-way valve 13b, while simultaneously the first one-way valve 13a and the third one-way valve 13c are closed to prevent flow of the first fluid 111 through the delivery device 11.

In some embodiments of the valve arrangement 13, the cracking (threshold) pressures of the first, second, third and fourth one-way valves 13a-13d are set at predetermined values to minimize and/or prevent free-flow of the first and second fluids 111,112. The threshold of each of the first, second, third and fourth one-way valves 13a-13d can be selectively established when the delivery device 11 is made. For example, the threshold of the valve can be established by selecting one or more of: a thickness of the flexible membrane 13e, the surface area of the flexible membrane 13e, and the geometry of the slits (e.g., length and/or shape) of the first, second, third and fourth one-way valves 13a-13d.

In one such embodiment, each of the first and fourth one-way valves 13a,13d have a zero cracking pressure, and each of the second and third one-way valves 13b,13c have a cracking pressure greater than zero bar (e.g., about 0.1 bar to about 10.0 bar, such as about 0.3 bar).

In another such embodiment, each of the first and fourth one-way valves 13a,13d have a cracking pressure greater than zero bar (e.g., about 0.1 bar to about 10.0 bar, such as about 0.3 bar), and each of the second and third one-way valves 13b,13c have a zero cracking pressure.

In another such embodiment, each of the first and second one-way valves 13a,13b have a zero cracking pressure, and each of the third and fourth one-way valves 13c,13d have a cracking pressure greater than zero bar (e.g., about 0.1 bar to about 10.0 bar, such as about 0.3 bar).

In another such embodiment, each of the first and second one-way valves 13a,13b have a cracking pressure greater than zero bar (e.g., about 0.1 bar to about 10.0 bar, such as about 0.3 bar), and each of the third and fourth one-way valves 13c,13d have a zero cracking pressure.

In yet another such embodiment, only one of the first, second, third and fourth one-way valves 13a-13d has a zero cracking pressure, and the other three of the first, second, third and fourth one-way valves 13a-13d have a cracking pressure greater than zero bar (e.g., about 0.1 bar to about 10.0 bar, such as about 0.3 bar).

In yet another such embodiment, each of the first, second, third and fourth one-way valves 13a-13d have a cracking pressure greater than zero bar (e.g., about 0.1 bar to about 10.0 bar, such as about 0.3 bar).

As noted above, some embodiments of the system 10 comprise an anti-free flow mechanism separate from the valve arrangement 13, for example the fifth and sixth one-way valves 13f,13g upstream from the valve arrangement 13 and/or the seventh one-way valve 13h downstream from the valve arrangement 13. In these embodiments, one or more of the first, second, third and fourth one-way valves 13a-13d (e.g., one, two, three or all four of these one-way valves) can have a zero cracking pressure, and the one or more one-way valves in the separate anti-free flow mechanism have a cracking pressure greater than zero bar (e.g., about 0.1 bar to about 10.0 bar, such as about 0.3 bar). For example, the fifth and sixth one-way valves 13f,13g can be present simultaneously and can have a cracking pressure greater than zero bar (e.g., about 0.1 bar to about 10.0 bar, such as about 0.3 bar). As another example, the seventh one-way valve 13h can be present and can have a cracking pressure greater than zero bar (e.g., about 0.1 bar to about 10.0 bar, such as about 0.3 bar).

In a preferred embodiment, the pumping by the pump 12 in a first direction decreases the pressure of the second chamber 15 (e.g., by suction) while the second pump passage 12b simultaneously increases the pressure of the fourth chamber 17 (e.g., by discharge), and the pumping by the pump 12 in a second direction opposite to the first direction increases the pressure of the second chamber 15 (e.g., by discharge) while the second pump passage 12b simultaneously decreases the pressure of the fourth chamber 17 (e.g., by suction). This configuration allows sequential administration of the first and second fluids 111,112 while the valve arrangement 13 prevents free-flow. In an embodiment, the control unit 12d is configured to control the direction of pumping by the pump 12. Preferably, stopping the pump 12 ceases flow in both the first and second flow paths.

In an embodiment in which the motor 12c of the pump 12 uses a shaft, the shaft can rotate one direction such that the first pump passage 12a decreases the pressure in the second chamber 15 and the second pump passage 12b simultaneously increases the pressure in the fourth chamber 17. The shaft of the motor 12c can rotate the opposite direction such that the first pump passage 12a increases the pressure in the second chamber 15 and the second pump passage 12b simultaneously decreases the pressure in the fourth chamber 17. As noted above, the rotation of the motor 12c can be controlled by the control unit 12d. However, as previously set forth, the present disclosure is not limited to a specific embodiment of the pump 12, and the pump 12 can operate using a mechanism alternative or additional to rotation.

Therefore, the system 10 can safely provide sequential administration of the first fluid 111 and the second fluid 112 to the patient 100. Moreover, the system 10 may be operated in various modes. For example, the system 10 may initially flush the delivery device 11 with a flushing solution and then switch to a feeding liquid. At selected intervals, the system 10 may then switch back to the flushing solution for a short period to flush the delivery device 11 to reduce the probability of blockages. As another example, the system 10 may deliver a predetermined amount of the first fluid 111 to the patient 100 and then deliver a predetermined amount of the second fluid 112 to the patient 100. As yet another example, the system 10 may intermittently deliver a predetermined amount of the first fluid 111 to the patient 100 and then deliver a predetermined amount of the second fluid 112 to the patient 100 in repeated cycles.

Another aspect of the present disclosure is a method of sequentially administering a first fluid and a second fluid to a patient using a single pump connected to a housing of a delivery device. The method can use the system 10 and/or the delivery device 11 disclosed herein or any other system or delivery device capable of performing the steps of the method. Preferably at least one of the first and second fluids is an enteral feeding formulation.

The delivery device can comprise first, second, third and fourth one-way valves within a housing, and the method can comprise operating the pump in one pumping direction to direct the first fluid from a first container in a first flow path through the delivery device and out of an administration port of the housing of the delivery device, the first flow path comprising the first one-way valve and the third one-way valve. The method can further comprise reversing the pumping direction of the pump to cease movement of the first fluid through the delivery device and direct the second fluid in a second flow path through the delivery device and out of the same administration port of the delivery device that the first fluid used to exit the delivery device, the second flow path comprising the second one-way valve and the fourth one-way valve.

The one-way valves prevent the second fluid from moving through the delivery device while the first fluid is pumped through the delivery device, and the one-way valves prevent the first fluid from moving through the delivery device while the second fluid is pumped through the delivery device. Specifically, the one-way valves prevent the second fluid from moving through the second flow path while the first fluid is pumped through the first flow path, and the one-way valves prevent the first fluid from moving through the first flow path while the second fluid is pumped through the second flow path.

The first, second, third and fourth one-way valves can be positioned in a flexible membrane that is positioned at least partially within the housing of the delivery device. Preferably the flexible membrane is one single piece, for example a single piece of material or a plurality of pieces of material connected together. In an embodiment, each of the first, second, third and fourth one-way valves are a slit valve.

Preferably the pumping direction of the pump is reversed automatically according to instructions stored within a control unit operatively connected to the pump. The method preferably does not include disconnection of tubing, does not include changing of pumps, and does not include manual adjustment of any valves. In an embodiment, the pumping direction of the pump is reversed at least twice. For example, the method can further comprise reversing the pumping direction of the pump an additional time to cease movement of the second fluid through the delivery device and again direct the first fluid in the first flow path.

The housing of the delivery device can comprise a first chamber, a second chamber, a third chamber, a fourth chamber, and an administration chamber. The first flow path of the first fluid can comprise the first chamber, then the second chamber, then through the pump to the fourth chamber, then into the administration chamber. The second flow path of the second fluid can comprise the third chamber, then the fourth chamber, then through the pump to the second chamber, then into the administration chamber. The first one-way valve can fluidly connect the first chamber to the second chamber, the second one-way valve can fluidly connect the second chamber to the administration chamber, the fourth one-way valve can fluidly connect the third chamber to the fourth chamber, and the third one-way valve can fluidly connect the fourth chamber to the administration chamber.

The method can comprise one or more of: directing the first fluid from a first container, through a first inlet port, in the housing of the delivery device into the first chamber; directing the first fluid from the first chamber, through the first one-way valve, into the second chamber only once a pressure differential over the first one-way valve exceeds a first threshold pressure of the first one-way valve; directing the first fluid from the second chamber through the pump to the fourth chamber by operation of the pump; directing the first fluid from the fourth chamber, through the third one-way valve, into the administration chamber only once a pressure differential over the third one-way valve exceeds a second threshold pressure of the third one-way valve; and directing the first fluid from the administration chamber, through an administration port, into an administration tube leading to the patient.

The method can comprise one or more of: directing the second fluid from a second container, through a second inlet port in the housing of the delivery device, into the third chamber; directing the second fluid from the third chamber, through the fourth one-way valve, into the fourth chamber only once a pressure differential over the fourth one-way valve exceeds a third threshold pressure of the fourth one-way valve; directing the second fluid from the fourth chamber through the pump to the second chamber by operation of the pump; directing the second fluid from the second chamber, through the second one-way valve, into the administration chamber only once a pressure differential over the second one-way valve exceeds a fourth threshold pressure of the second one-way valve; and directing the second fluid from the administration chamber, through the administration port, into the administration tube leading to the patient.

Yet another aspect of the present disclosure is a method of making a delivery device configured to connect to a single pump and sequentially administer a first fluid and a second fluid to a patient. The method can make the delivery device 11 disclosed herein or any delivery device capable of being made by the steps of the method.

The method can comprise forming a housing that contains a first flow path and a second flow path that begin at different locations on the housing relative to each other and emerge through the same administration port. The method can further comprise positioning a first one-way valve, a second one-way valve, a third one-way valve and fourth one way valve within the housing such that the first one-way valve and the third one-way valve are positioned in the first flow path and the second one-way valve and the fourth one-way valve are positioned in the second flow path. Preferably the positioning of the first, second, third and fourth one-way valves within the housing comprises positioning a flexible membrane comprising the first, second, third and fourth one-way valves at least partially within the housing. In an embodiment, each of the first, second, third and fourth one-way valves are a slit valve.

In a preferred embodiment, the forming of the housing comprises connecting a first wall, a second wall, a third wall and a fourth wall such that the first wall, the third wall and the flexible membrane define at least part of a first chamber; the second wall, the third wall and the flexible membrane define at least part of a second chamber; the first wall, the fourth wall and the flexible membrane define at least part of a third chamber; the second wall, the fourth wall and the flexible membrane define at least part of a fourth chamber; the first wall and the flexible membrane define at least part of an administration chamber; the first, second and administration chambers define at least part of the first flow path; and the third, fourth and administration chambers define at least part of the second flow path.

Yet another aspect of the present disclosure is a method of making a system configured to sequentially administer a first fluid and a second fluid to a patient. The method comprises connecting to a pump any embodiment of a delivery device disclosed herein or to any delivery device made by a method disclosed herein. The pump can be a positive displacement pump. The pump can be connected to a first and second pump communication port of the delivery device. The pump can be configured to pump in a first direction and a second direction opposite to the first direction to alternate between (i) suction through the first pump communication port and discharge through the second pump communication port and (ii) suction through the second pump communication port and discharge through the first pump communication port.

The method can comprise connecting a first inlet port of the delivery device to a first tube that leads to a first container that holds the first fluid. The method can comprise connecting a second inlet port of the delivery device to a second tube that leads to a second container that holds the second fluid. The method can comprise connecting one or more outlet ports to an administration tube that leads to a patient.

Various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

1. A delivery device comprising a housing comprising a first inlet port, a second inlet port, an outlet port, a first pump communication port, and a second pump communication port, the delivery device further comprising a first one-way valve, a second one-way valve, a third one-way valve, and a fourth one-way valve;
a first flow path extends from the first inlet port through the first one-way valve and the third one-way valve to the outlet port; and
a second flow path extends from the second inlet port through the second one-way valve and the fourth one-way valve to the outlet port.

2. The delivery device of claim 1, comprising one or more flexible membranes at least partially positioned within the housing, and the one or more flexible membranes provide the first, second, third and fourth one-way valves.

3. The delivery device of claim 1, wherein the housing comprises a first chamber, a second chamber, a third chamber, a fourth chamber, and an administration chamber that define at least part of the first flow path.

4. (canceled)

5. The delivery device of claim 3, wherein the housing comprises:
a first wall that provides the first inlet port and the second inlet port; and
a second wall that provides the first pump communication port and the second pump communication port.

6-10. (canceled)

11. The delivery device of claim 1, wherein the first inlet port, the second inlet port, the outlet port, the first pump communication port and the second pump communication port are the only fluid connections on the housing of the delivery device.

12. (canceled)

13. The delivery device of claim 1, wherein each of the first and fourth one-way valves has a zero cracking pressure, and each of the second and third one-way valves has a cracking pressure greater than zero bar.

14. The delivery device of claim 1, wherein each of the first and fourth one-way valves has a cracking pressure greater than zero bar, and each of the second and third one-way valves has a zero cracking pressure.

15. The delivery device of claim 1, wherein each of the first, second, third and fourth one-way valves has a cracking pressure greater than zero bars.

16. The delivery device of claim 1, wherein only one of the first, second, third and fourth one-way valves has a zero cracking pressure, and three of the first, second, third and fourth one-way valves has a cracking pressure greater than zero bars.

17. The delivery device of claim 1, wherein each of the first and second one-way valves has a zero cracking pressure, and each of the third and fourth one-way valves has a cracking pressure greater than zero bar.

18. The delivery device of claim 1, wherein each of the first and second one-way valves has a cracking pressure greater than zero bar, and each of the third and fourth one-way valves has a zero cracking pressure.

19. A system comprising:
a delivery device comprising a housing comprising a first inlet port, a second inlet port, an outlet port, a first pump communication port and a second pump communication port, the delivery device further comprising a first one-way valve, a second one-way valve, a third one-way valve, and a fourth one-way valve; a first flow path extends from the first inlet port through the first one-way valve and the third one-way valve to the outlet port; a second flow path extends from the second inlet port through the second one-way valve and the fourth one-way valve to the outlet port; and
a pump comprising a first passage that leads to the first pump communication port and a second passage that leads to the second pump communication port.

20. The system of claim 19, wherein the pump is a single pump that is the only pump connected to the delivery device.

21. The system of claim 19 comprising:
a first component connected to the first inlet port by a first tube and selected from the group consisting of (i) a first container that holds a first fluid and (ii) a first connector configured to connect to a first container that holds a first fluid; and
a second component connected to the second inlet port by a second tube and selected from the group consisting of (i) a second container that holds a second fluid and (ii) a second connector configured to connect to a second container that holds a first fluid.

22. The system of claim 19 wherein the pump is configured to:
pump in a first direction to direct the first fluid through the first flow path while the second and fourth one-way valves are closed to prevent the second fluid from moving through the second flow path, and the first and third one-way valves are open, pump in a second direction opposite to the first direction to direct the second fluid through the second flow path while the first and third one-way valves are closed to prevent the first fluid from moving through the first flow path, and the second and fourth one-way valves are open, and
not pump in the first direction or the second direction, to prevent the first fluid from moving through the first flow path and the second fluid from moving through the second flow path.

23. The system of claim 19 comprising:
a fifth one-way valve upstream from the first inlet port; and
a sixth one-way valve upstream from the second inlet port,
wherein at least one of the first, second, third and fourth one-way valves has a zero cracking pressure, and each of the fifth and sixth one-way valves have a cracking pressure greater than zero bar.

24. The system of claim 19 comprising an additional one-way valve that is downstream from the outlet port, wherein at least one of the first, second, third and fourth one-way valves have a zero cracking pressure, and the seventh one-way valve has a cracking pressure greater than zero bar.

25. (canceled)

26. A method of sequentially administering a first fluid and a second fluid to a patient using a single pump connected to a housing of a delivery device, the delivery device comprising a first one-way valve, a second one-way valve, a third one-way valve, and a fourth one-way valve within the housing, the method comprising:
operating the pump in one pumping direction to direct the first fluid from a first container in a first flow path through the delivery device and out of an administration port of the housing of the delivery device, the first flow path comprising the first one-way valve and the third one-way valve, wherein the operating of the pump to direct the first fluid through the first flow path prevents the second fluid from flowing through the delivery device; and
reversing the pumping direction of the pump to cease movement of the first fluid through the delivery device and direct the second fluid in a second flow path through the delivery device and out of the same administration port of the delivery device that the first fluid used to exit the housing of the delivery device, the second flow path comprising the second one-way valve and the fourth one-way valve, wherein the operating of the pump to direct the second fluid through the second flow path prevents the first fluid from flowing through the delivery device.

27. The method of claim 26 wherein the reversing of the pumping direction is performed automatically by a control unit operatively connected to the pump, according to instructions stored within the control unit that specify a number of times to perform the reversing.

28. The method of claim 26 comprising reversing the pumping direction of the pump an additional time, to cease movement of the second fluid through the delivery device and again direct the first fluid in the first flow path.

29-36. (canceled)
